(19) United States
(12) Patent Application Publication
(10) Pub. No.: US 2008/0095798 A1
(43) Pub. Date: Apr. 24, 2008

(54) Ii-key enhanced vaccine potency

(76) Inventors: Robert Humphreys, Acton, MA (US); Douglas Macmillan Powell, Littleton, MA (US); John Zinckgraf, Sturbridge, MA (US)

Correspondence Address: Kevin M. Farrell;Pierce Atwood, Suite 350, One New Hampshire Avenue, Portsmouth, NH, 03801, US

(21) Appl. No.: 11/582,596

(22) Filed: Oct. 18, 2006

(51) Int. Cl. A61K 48/00; A61K 39/12; A61K 39/02

(52) U.S. Cl. 424/204.1; 424/234.1; 514/44

(57) ABSTRACT

Disclosed is a method for increasing vaccine potency whereby a subject's immune system is first primed with an Ii-Key hybrid peptide construct before the subject subsequently receives a vaccine for a pathogen of interest. The vaccine may be comprised of a protein or portion thereof that is encoded by the genome of the pathogen. The vaccine may also be a DNA vaccine comprised of DNA encoding a protein of the pathogen. The Ii-Key hybrid peptide construct includes the LRMK residues of Ii-Key protein and an MHC Class II epitope of the protein or portion thereof which is used in the vaccine. The Ii-Key construct may be administered in the form of a nucleic acid construct encoding the Ii-Key hybrid peptide. Priming with Ii-Key peptides enhances the immunogenicity of rHA protein and HA and HIV DNA vaccines. Methods are described relating to the use of Ii-Key hybrid constructs in vaccine protocols wherein the pathogen is HIV or Influenza A, including H5N1. Methods and compositions are described wherein the MHC Class II epitope of the Ii-Key hybrid is hemagglutinin encoded by Influenza A or the Gag protein encoded by HIV.

### BACKGROUND OF THE INVENTION

The immune system responds to foreign pathogens, tumor cells, allergens, autoimmune disease-inducing processes, and grafts by recognizing ‘foreign’ or ‘abnormal’ structures as antigens. Most antigens are proteins, either synthesized by host cells, or by pathogens. Such antigens are processed (proteolytically digested) into peptide fragments. The fragments are then presented in a peptide-presenting structure on the surface of an antigen presenting cell (APC). These peptide presenting structures are called major histocompatibility complex (MHC) molecules, so named because they were first recognized as products of polymorphic genes belonging to the MHC gene cluster. The MHC genes control many activities of immune cells, such as graft rejection and the killing of pathogen-infected cells by specific killer T lymphocytes.

The immune response to a specific antigen is mediated by T lymphocytes which respond when fragments of the antigen are presented on the APC's surface. Within an APC, peptide fragments of a proteolytically processed antigen become bound in the antigenic-peptide binding site of MHC molecules. These peptide-MHC molecule complexes are then transported to the cell surface for recognition (of both the foreign peptide and the adjacent surface of the presenting molecule) by the T cell receptors on responding T lymphocytes. This antigen-specific recognition event initiates the immune response cascade which leads to a protective immune response, or in the case of autoimmune processes, a deleterious immune response.

There are two classes of MHC molecules: MHC class I and MHC class II. MHC Class I molecules are synthesized in the endoplasmic reticulum. They receive peptides exclusively from endogenously synthesized proteins, such as from a virus, and present them to CD8+ cytotoxic T-lymphocytes (CTLs), which then become activated and can directly kill the antigen presenting cell. MHC class II molecules are also synthesized in the endoplasmic reticulum. When synthesized, their antigenic peptide binding sites are blocked by the invariant chain (Ii) protein. The Ii protein prevents MHC Class II molecules form binding endogenous antigenic peptides which have formed in the cytoplasm and been transported into the endoplasmic reticulum. Complexes of MHC class II molecules and Ii protein are transported from the endoplasmic reticulum to a post-Golgi compartment where Ii is released by proteolysis after which a specific antigenic peptide binds to the MHC class II molecule. MHC class II molecules bind only exogenous antigens, internalized via endocytosis. MHC class II molecules present antigens to CD4+ helper T-lymphocytes (T helper cells). Once activated, T helper cells help activate cytotoxic T lymphocytes (Killer T cells) and B lymphocytes. (Blum et al., Proc. Natl. Acad. Sci. USA 85: 3975 (1988); Riberdy et al., Nature 360: 474 (1992); Daibata et al., Mol. Immunol. 31: 255 (1994); Xu et al., Mol. Immunol. 31: 723 (1994); Xu et al., Antigen Processing and Presentation, Academic Press, NY p227 (1994); Kropshofer et al., Science 270: 1357 (1995); and Urban et al., J. Exp. Med. 180: 751 (1994)).

R. Humphreys' (1996) U.S. Pat. No. 5,559,028, and Humphreys' et al. (1999) U.S. Pat. No. 5,919,639 revealed the mechanisms by which Ii protein is cleaved, where fragments released in the course of cleavage go on to regulate the binding and locking in of antigenic peptides within the antigenic peptide binding site of MHC class II molecules (Adams et al., Eur. J. Immunol. 25: 1693 (1995); Adams et al., Arzneim. Forsch./Drug Research 47: 1069 (1997); and Xu et al., Arzneim. Forsch./Drug Research in press (1999)). The referenced patents, furthermore, disclosed novel therapeutic compounds and methods to control this initial regulatory, antigenic peptide-recognizing event. The identification of these mechanisms opened new avenues of therapeutic intervention.

U.S. application Ser. No. 09/396,813 (now U.S. Pat. No. 6,432,409) and Ser. No. 11/033,039 disclose hybrid peptides useful in connection with modulation of the immune system. The disclosures are based on the discovery that an MHC Class II-restricted antigenic epitope which is covalently linked to a mammalian Ii-Key peptide by an appropriate intervening chemical structure, forming a hybrid polypeptide, is presented to T lymphocytes by antigen presenting cells with significantly higher efficacy than is the precursor antigenic epitope. The disclosures of U.S. Pat. No. 6,432,409 and U.S. application Ser. No. 11/033,039 are incorporated herein by reference.

Two prominent pathogens currently of high concern are the HIV and influenza viruses, particularly the H5N1 avian flu virus. HIV has infected millions of people throughout the world and many fear that H5N1 will do the same. H5N1 is a deadly virus to humans, birds, and several other animal species. In the event of an H5N1 or other epidemic, health care providers will face a vast shortage of vaccine supplies. As many as 200 million people in the United States could become infected, with up to 2 million deaths (Poland, G. A., N Engl J Med 354:141 (2006)). Over a billion doses of vaccine may be needed to stop a pandemic. Egg-based vaccines for seasonal flu are unlikely to provide cross-protection against H5NI. As of 2006, there are more than 30 H5N1 vaccine candidates being clinically tested (Poland, G. A., N Engl J Med 354:141 (2006)), many employing egg-based manufacture. Because of the high pathogenicity of H5N1, the manufacture of H5N1 vaccines using embryonated chicken eggs typically yields low viral titers. Other H5N1 vaccines currently being evaluated include adenoviral vectored antigens (Gao, W. et al., J Virol 80:1959 (2006)), DNA vaccines (Bright, R. A. et al., Virology 308:270 (2003), Epstein, S. L. et al., Emerg Infect Dis 8:796 (2002)) and vaccines using cell culture-based approaches. Studies have shown that known H5N1 vaccines are poorly immunogenic, requiring doses up to 12-fold greater than seasonal flu vaccine (Bresson, J. L. et al., Lancet 367:1657 (2006); Treanor, J. J. et al., N Engl J Med 354:1343 (2006); Treanor, J. J., et al., Vaccine 19:1732 (2001); Stephenson, I. et al., J Infect Dis 191:1210 (2005)).

Studies have shown that activating both arms of the immune system provides the most effective anti-viral response, usually relying heavily on the action of CD4+ T cells. The cytokines released from activated CD4+ T cells indirectly aid B cells and CD8+ T cells, while providing essential support for the induction of memory B and T cells (Brown, D. M. et al., Semin Immunol 16:171 (2004); Swain, S. L. et al., Immunol Rev 211:8 (2006)). CD4+ T cells have direct roles in the control of viral infections (Hogan, R. J. et al., J Exp Med 193:981 (2001); Paludan, C. et al., J Immunol 169:1593 (2002)), including affecting influenza-specific cytolytic activity (Graham, M. B. et al., J Exp Med 180:1273 (1994); Graham, M. B., and T. J. Braciale, J Exp Med 186:2063 (1997)). Since the contribution of each cell type in protecting humans against H5N1 infection is unknown, vaccines designed to induce multiple arms of the immune system and generate broad immunity will likely be the most effective against an H5N1 pandemic.

The application of peptide vaccines for the induction of broad-based immunity against influenza viruses has previously been investigated. Studies have shown that the inclusion of B, T-helper, and CTL influenza-derived epitopes as a peptide vaccine can stimulate strong immunity and protection from viral challenge (Horvath, A. et al., Immunol Lett 60:127 (1998); Westerfeld, N., and R. Zurbriggen. J PPept Sci 11: 707 (2005); Bianchi, E. et al, J Virol 79:7380 (2005); Ninomiya, A. et al., Vaccine 20: 3123 (2002); Brumeanu, T. D. et al., J Virol 71:5473 (1997); Jeon, S. H. et al., Vaccine 20:2772 (2002); Simeckova-Rosenberg, J. et al., Vaccine 13:927 (1995)). Other studies have shown that protective and cross-strain immunity can be induced by including highly conserved epitopes (Levi, R.and R. Arnon. Vaccine 14:85 (1996); Ben-Yedidia, T. et al., Mol Immunol 39:323 (2002)).

Peptide vaccines aimed at antigen-specific CD4+ T cell stimulation have the potential to protect against serologically distinct viral strains. The focus of influenza vaccine development classically has been on the induction of neutralizing antibodies. This technique is not likely to be effective if a strain emerges that is distinct from that used to generate the vaccine. Several murine studies have shown that CD4+ cells are instrumental in orchestrating an effective anti-viral immune response (Brown, D. M. et al., Semin Immunol 16:171 (2004; Swain, S. L. et al., Immunol Rev 211:8 (2006)). Swain et al. have demonstrated that adoptive transfer of PR8 influenza-specific Th1 polarized effector cells into syngeneic hosts can protect mice from lethal challenge, while mice not receiving primed CD4+ cells all succumbed to death (Swain, S. L. et al., Immunol Rev 211:8 (2006)). These experiments emphasize the importance of CD4+ T cells in the control of viral infection.

Addressing some of the inadequacies of current H5N1 vaccines, new methods and compositions described herein offer the promise of stretching the current vaccine supply and reducing the dosage needed to vaccinate the population.

### SUMMARY OF THE INVENTION

In one aspect, the present invention relates to a method to improve the potency of DNA and peptide vaccines containing MHC Class II-presented epitopes of antigens of interest. The present invention involves priming the immune system of a subject with Ii-Key hybrid peptides such that the potency of a subsequently administered DNA or peptide vaccine is augmented. The Ii-Key construct may be administered in the form of a nucleic acid construct encoding the Ii-Key hybrid peptide.

Two examples are the use of Ii-Key antigenic epitope hybrids in vaccination protocols to protect against HIV and Influenza A, particularly H5N1. By first priming naive T-helper cells with a hybrid protein comprised of Ii-Key and a highly conserved MHC class II epitope derived from the HA protein, the immunological response to a clinically tested rHA vaccine used in the prevention and treatment of H5N1 is improved. By priming a subject's immune system with these hybrid peptides before boosting with a DNA or protein vaccine, limited supplies of vaccines can be extended.

In another aspect, this invention relates to compositions used to increase the potency of DNA and peptide vaccines by priming the subject's immune response. The compositions are hybrid peptides comprised of the LRMK amino acid residues of the Ii-Key protein and an MHC class II epitope, wherein the epitope is a hemagglutinin encoded by the H5N1 strain of influenza A or a Gag protein encoded by HIV.

### BRIEF DESCRIPTION OF THE DRAWINGS

FIG. 1. In vitro IFN-γ and IL-4 ELISPOT responses following vaccination. BALB/c mice (5/group) were immunized at week 0 either subcutaneously with Ii-Key peptide (100 μg) emulsified in RIBI adjuvant or intramuscularly with HA DNA vaccine (50 μg). Two weeks later, mice were boosted with HA DNA. Twelve days post-boost mice were sacrificed and spleens aseptically removed. Pooled splenocytes were used in an in vitro peptide restimulation assay with the Ii-Key peptides indicated on the X-axis. Control mice received an irrelevant DNA vaccine (B5R) or RIBI/PBS. Results indicate the geometric mean of samples assayed in triplicate.

FIG. 2. In vitro IFN-γ ELISPOT responses following vaccination. BALB/c mice (4/group) were immunized at week 0 either subcutaneously with 100 μg Ii-Key peptide (Gag298 or Gag198) emulsified in CFA adjuvant or intramuscularly with pcDNA/SynGag vaccine (50 μg). Nine days later, all mice were boosted with pcDNA/SynGag. One group of mice was boosted a second time at day 16. Eleven days post-boost mice were sacrificed and spleens aseptically removed. Pooled splenocytes were used in an in vitro peptide restimulation assay with the Ii-Key peptides indicated on the X-axis. Results indicate the geometric mean of samples assayed in triplicate.

FIG. 3. IFN-γ/IL-4 responses following rHA immunization. Mice were immunized subcutaneously at day 0 with 10 μg of rHA emulsified in CFA adjuvant. Mice were boosted at day 14, followed by in vitro splenocyte restimulation (72 hr) two weeks post-boost. Data represents the mean±SEM of peptides assayed in triplicate.

FIG. 4. IFN-γ/IL-4 responses following DNA, immunization. Mice were immunized intramuscularly at day 0 with 50 μg of pcDNA/HA and boosted at day 14, followed by in vitro splenocyte restimulation (72 hr) 10 days post-boost. Data represents the mean±SEM of peptides assayed in triplicate.

FIG. 5A. IFN-γ ELISPOT responses following homologous Ii-Key hybrid peptide immunization. Mice were immunized subcutaneously at day 0 with 100 μg of Ii-Key peptide emulsified in RIBI adjuvant. Mice were boosted at day 14, followed by in vitro splenocyte restimulation (72 hr) two weeks post-boost. The Ii-Key peptides used in restimulation are indicated on the X-axis. Data represents the mean±SEM of peptides assayed in triplicate.

FIG. 5B. IFN-γ ELISPOT responses following heterologous immunization. Mice were immunized subcutaneously at day 0 with 100 μg of Ii-Key peptide, 33 μg/peptide (black bar) of Ii-Key peptide, or 10 μg rHA emulsified in RIBI adjuvant, followed by boosting at day 21 with 10 μg rHA. In vitro restimulation (72 hr) was performed two weeks post-boost. The Ii-Key peptides used in restimulation are indicated on the X-axis. Data represents the mean±SEM of peptides assayed in triplicate. *P<0.001 compared to RIBI/rHA and rHA/rHA, **P<0.05 compared to RIBI/rHA and rHA/rHA. AEO=Antigenic Epitope Alone.

FIG. 6. IFN-γ ELISPOT responses following heterologous immunization and CD4+ depletion. Mice were immunized subcutaneously at day 0 with either 100 μg of Ii-Key peptide, 33 μg/peptide (black bar) of Ii-Key peptide, or 10 μg rHA emulsified in RIBI adjuvant, followed by boosting at day 21 with 10 μg rHA. In vitro restimulation (48 hr) was performed two weeks post-boost. The Ii-Key peptides used in restimulation are indicated on the X-axis. Data represents the mean±SEM of peptides assayed in triplicate.

FIG. 7. Mouse IgG anti-HA responses. Mice were immunized as described in FIG. 5B, with serum collected at the time of sacrifice. End point titrations were performed by testing anti-sera against rHA in an indirect ELISA. Results represent the mean±SEM of samples tested in duplicate. *P<0.001 compared to all other groups.

### DETAILED DESCRIPTION OF THE INVENTION

The present invention relates to the discovery that priming the immune system with MHC class II Ii-Key hybrid peptide vaccines followed by a DNA or peptide vaccine booster increases the T cell response of the subject. By first priming with an Ii-Key hybrid, followed by a DNA vaccine booster, the subject's resultant T cell response is equivalent to that of 2-3 DNA vaccinations. Stronger T cell responses correlate with increased protection from viral infection and contribute to long-term immunological memory. Mice primed with Ii-Key hybrids and boosted with rHA protein vaccine exhibited IFN-γ responses greater than 20 fold higher than mice receiving one dose of protein vaccine. By substituting Ii-Key hybrid peptide vaccines for some of the doses of a DNA or peptide vaccine series, vaccine supply shortages can be avoided.

### DEFINITIONS

The following bolded terms are used throughout this document with the following associated meanings:

Antigen-presenting cell (APC): a cell that displays a foreign antigen complexed with a MHC molecule on its surface.

BALB/c: a popular inbred mouse strain used in many different research disciplines, but most often in the production of monoclonal antibodies. The Balb/c mouse is albino and small in size. A BALB/c(H-2^{d}) mouse differs from the BALB/c strain by the H-2^{d} allele, an allele of the MHC Complex.

Bystander effect: the suppression or enhancement of the immune response to one antigen that occurs when that one antigen is presented in the presence of a second antigen to which tolerance is already established.

CFA: Complete Freund's Adjuvant.

Codon Optimization: the adjustment of the codon frequency of a foreign protein to match that of its host's expression system. Some species of organisms prefer certain codons over other synonymous codons. Translation can be made more efficient by using the codon preferred by the target organism.

CTL: cytotoxic T lymphocytes; killer T lymphocytes or cells.

Cytokine: intercellular signals; peptides released from one cell that affect another cell's behavior.

ELISPOT: an ELISA-like assay where the T lymphocytes are placed in a microtiter well coated with cytokine-specific antibodies; most often used to measure the number of antigen specific T cells in a sample. CD4 responses are measured by IL-4 capture and by IFN-γ capture.

IFA: Incomplete Freund's Adjuvant.

Ii-Key: the immunoregulatory motif within the Ii protein.

Interferon (IFN): a glycoprotein produced by the cells of the immune systems of most animals in response to challenges by foreign agents; a cytokine.

Interleukin (IL): a protein secreted by macrophage and T-lymphocytes that induces growth/differentiation of lymphocytes; a cytokine.

Lymphocyte: a type of leukocyte. Lymphocytes may be B cells or T cells.

Major histocompatibility complex (MHC): a large gene family found in most vertebrates which plays an important role in the immune system.

pcDNA: a plasmid-based cloning and expression vector manufactured by Invitrogen™.

Promiscuous peptide: a peptide which is presented by more than one human leukocyte antigen-diversity region allele.

RIBI: an oil-in-water emulsion composed of Monophosphoryl Lipid A and Trehalose Dicorynomycolate. RIBI can be used as an alternative to Freund's adjuvant.

Syn-: synthetic.

### Embodiments

As discussed in the Background section, a vaccine supply shortage may result in large numbers of casualties in the United States and abroad. Methods of reducing the dosage needed to protect the population and increase the potency of existing vaccines are needed. Although effective immunization for some pathogenic organisms can be achieved through immunization with recombinantly-produced proteins, or even synthetic peptides, for others it has been necessary to produce the virus itself, isolate the virus and immunize using a heat-killed or chemically-inactivated form of the virus. This is true, for example, in connection with the influenza virus. Although much of the following discussion relates specifically to influenza virus, the principles discussed are broadly applicable to viruses, and pathogens generally.

Vaccine directed against seasonal influenza virus is typically produced by inoculating an embryonated chicken egg and allowing the virus to propogate. Alantoic fluid is then collected from the egg and the virus is heat-killed or chemically inactivated prior to immunization. In contrast to seasonal influenza vaccines, the H5N1 pandemic influenza strain typically yields low viral titers using embryonated chicken eggs, primarily due to the virus' high pathogenicity. Coupled with unusually low immunogenicity of the vaccine (requiring much higher doses), it is anticipated that far fewer vaccine doses can be generated using this means than would be required to combat a pandemic. It is estimated that 200 million people in the United States could become infected, with 90 million becoming clinically ill and up to 2 million deaths (Poland, N. Engl. J. Med. 354: 1411 (2006).

Stockpiling millions of vaccine doses has limited utility because of vaccine half-life and makes little sense due to the potential emergence of mutant strains through antigenic drift, rendering such vaccines ineffective. Although reverse genetic vaccines can improve the efficiency of virus propagation in chicken eggs and induce protection following challenge, their clinical evaluation has been delayed by regulatory concern. Other H5N1 vaccines in development are being evaluated including adenoviral vectored antigens, DNA vaccines, and vaccines using cell culture-based approaches, but their introduction into the clinic has been delayed.

While several different vaccine candidates have been tested in the clinic, the results have been variable and often disappointing. Most recently, a recombinant inactivated split-virion vaccine was tested in which participants were injected with 1.5, 15 or 30 mg/dose of hemagglutinin with or without alum adjuvant. Based on Hemagglutination Inhibition (HI) and microneutralization analysis, vaccine recipients receiving the highest dose yielded the highest responses, while lower doses resulted in suboptimal titers. In a similar study, vaccine recipients were immunized with varying doses of an egg-based, non-adjuvanted recombinant H5 virus (rgA/Vietnam/1203/2004×A/PR/8/34). In that study, only 56% of people immunized with two doses of 90 μg had HI titers that exceeded pre-immune titers by at least four-fold, implying the vaccine was poorly immunogenic (Treanor et al., N. Engl. J. Med. 354: 1343 (2006)). Similar findings were observed in a study in which baculovirus-derived H5N1 hemagglutinin was used as the vaccine candidate (Treanor et al., Vaccine 19: 1732 (2001)). Such high doses (up to 12-fold greater than seasonal flu vaccine), which induce only modest immunogenicity, may not provide adequate protection in the event of a pandemic, making it difficult to rapidly produce enough vaccine for adequate population coverage. In addition, the level of cross-protection, if any, afforded by these recombinant vaccines towards other H5N1 strains has not been determined. Nonetheless, these studies provide important information about the safety and efficacy of such vaccines.

Influenza infection and vaccine development has been most thoroughly investigated in murine model systems. Studies have shown that a lack of B cells in mice can lead to increased mortality following viral challenge (Mozdzanowska et al., Virology 239:217 (1997); Mozdzanowska et al., J. Virol. 79: 5943 (2005)), implicating the importance of having strong anti-viral humoral immunity, although the induction of CD8+ effector responses also contributes to viral clearance and recovery (Topham et al., J. Immunol 159: 5197 (1997)). It has been shown that activation of both arms of the immune system yields the most effective anti-viral response, and in most instances, relies heavily on the aid of CD4+ T cells. The cytokine milieu released from activated CD4+ T cells provides indirect “help” for B cells and CD8+ T cells, as well as providing essential support for the induction of memory B and T cells (Brown et al., Semin. Immunol. 16: 171 (2004); Swain et al., Immunol. Rev. 211:8 (2006)). Additional effector functions have been described for CD4+ T cells in the direct control of viral infections including influenza-specific cytolytic activity. Studies have also shown that CD4-depleted mice can clear the highly lethal mouse influenza PR8 virus, although the combination of CD4+, CD8+ and B cells has been shown to be associated with increased viral clearance and survival in mice, suggesting a multi-pronged induction is most efficient for protection. The contribution of each cell type in protecting humans against H5N1 infection is currently unknown and may depend in large part on the pathogenicity and overall virulence of the circulating strain. Taken together, H5N1 vaccines designed to induce multiple arms of the immune system and generate broad immunity will likely be the most effective against an H5N1 pandemic.

To address some of the shortcomings of current H5N1 vaccines, a novel H5NI influenza vaccine was developed that can improve the immunogenicity and allow for dose-sparing when used in combination with other H5N1 vaccines. Using highly conserved class II epitopes to prime naive T-helper cells, this approach was shown to improve the immunological response to a clinically tested rHA vaccine. Using the SYFPEITHI program (Rammensee et al., Immunogenetics 50: 213 (1999)), which allows for selection of HLA-restricted class II epitopes, 24 MHC class II epitopes were identified which are derived from the HA protein sequence predicted on a cumulative basis to interact with six of the most common human HLA-DR alleles. These epitopes were modified to include a segment of the immunoregulatory Ii protein (termed Ii-Key), which permits for extracellular MHC class II loading of peptides, thereby bypassing the need for exogenous processing and presentation. Such Ii-Key peptides enhance in vitro T cell responses (Adams et al., Eur. J. Immunol. 25: 1693 (1995)) as well as in vivo T and B cell responses to several vaccines under development (Kallinteris et al., Vaccine 21: 4128 (2003); Kallinteris et al., J. Immunother. 28: 352 (2005); Kallinteris et al., Frontiers in Bioscience:46 (2006)). Extracellular peptide loading may also more quickly activate naive T cells as demonstrated by others (Bot et al., J. Immunol 157: 3436 (1996)). One such vaccine generated using an MHC class II epitope from the Her2/neu protein is currently in clinical trials and has shown the ability to generate robust T-helper cell responses in patients (Mittendorf et al., Journal of Clinical Oncology, 2006 ASCO Annual Meeting Proceedings Part I. 24: 2532 (2006)). Although Ii-Key peptide vaccines may provide partial protection as a “stand-alone” against H5N1, they are primarily intended to augment the response to other vaccines, such as rHA used in this study, while at the same time decreasing the dose needed to induce strong immunity. Thus priming with peptides can be reasonably expected to extend the potentially limited supplies of other vaccine types, including rHA. Priming of CD4 T cells using peptides has been shown previously to increase the immune response to protein vaccination (Hosmalin et al., J. Immunol 146: 1667 (1991)). In those studies, rhesus monkeys were primed with three T cell epitopes derived from HIV gp120 and gp41, followed by boosting with recombinant gp 160 protein. Compared to control animals which were not primed with peptides, there was a significant improvement in both T cell proliferation and antibody response, indicative of peptide induced T-helper activity. Other studies have also observed improved immunogenicity following peptide priming (Vaslin et al., AIDS Res. Hum. Retroviruses 10: 1241 (1994)). It was hypothesized that priming mice with modified H5N1 class II epitopes, followed by boosting with rHA would improve the T and B cell response compared to rHA alone. As demonstrated in the Exemplification section which follows, the results show that priming with Ii-Key/HA epitope hybrids improves the immunogenicity of rHA and indicate a role for these types of peptides as a dose-sparing strategy for other H5NI vaccines in development.

Thus, in one aspect, the present invention relates to a dose-sparing method for increasing the potency of a vaccine directed toward a pathogen of interest in a subject. A vaccine is provided in connection with this method. The vaccine can include, for example, traditional heat-killed or chemically inactivated virus. Alternatively, the vaccine can include isolated protein from the pathogen of interest, or fragments thereof. The vaccine can also include protein or peptides produced by recombinant DNA techniques, or synthetic peptides. The present invention includes methods of increasing vaccine potency wherein the pathogen of interest is a virus or a bacterium. More specifically, the present invention includes methods wherein the pathogen is an HIV or influenza virus, including the H5N1 strain of influenza. Since, as discussed above, this vaccine material is often in limited supply, it is desirable to increase the potency of the vaccine so that the limited supply is effective for the immunization of as many individuals as possible.

The results disclosed herein demonstrate that the use of an Ii-key hybrid construct to prime the immune system of the subject prior to administration of the vaccine is surprisingly effective in increasing the potency of the vaccine relative to a non-primed administration. The Ii-key sequence has been described herein, and in the prior art. The Ii-key construct utilized in connection with the present invention includes at least the LRMK residues of the Ii-key sequence joined, through a linker, to an MHC class II epitope which is found within the hybrid construct discussed above. The linker is sized to provide spacing between the Ii-key element and the MHC class II epitope which results in maximal enhancement of the immune response. Generally speaking, this spacer provides spacing between these elements that approximates the spacing that would be provided by an amino acid sequence of 15-25 amino acid residues. The linker need not be comprised of amino acids, although this composition does simplify production of the hybrid construct. Alternatives to the amino acid linker portion have been described in the prior art.

The immune system of the subject is primed using an Ii-key construct of the type described above. Generally speaking, the Ii-key hybrid construct is formulated for injection. This formulation includes a physiologically compatible buffer and, optionally, an adjuvant. Many adjuvants are known in the art and the selection of one adjuvant over another is a matter of routine experimentation. Typically, the administration of the Ii-key construction formulation is by intramuscular or subcutaneous injection.

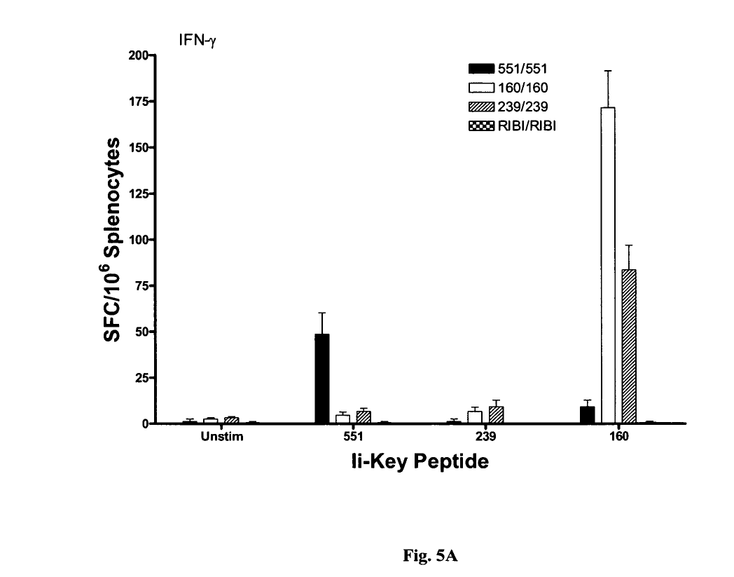

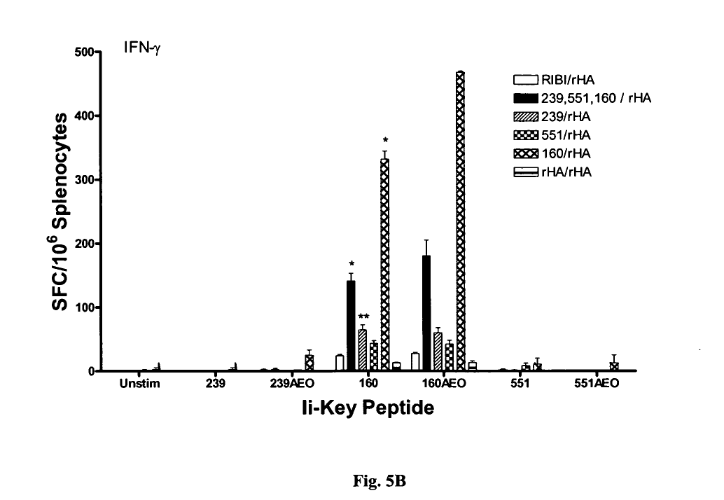

Following a period of time sufficient for the immune system of the organism to respond to the Ii-key hybrid administration, the vaccine composition is administered. Like the Ii-key formulation, the vaccine composition is typically administered in a physiologically compatible buffer with, or without, an adjuvant. The results shown below demonstrate a remarkable enhancement in the potency of influenza and HIV vaccine compositions following priming with an Ii-key hybrid construct. Results detailed in the Influenza DNA Vaccine Experiment of the Exemplification section show that T cell activity was greater in mice primed with an Ii-Key hybrid followed by a booster of DNA vaccine than in mice given two DNA vaccine doses. Likewise, the results of the HIV DNA Vaccine Experiment detailed in the Exemplification show that mice primed with an Ii-Key hybrid and boosted with DNA vaccine exhibited twice as much T cell activity as mice that were primed and boosted with DNA vaccine. The data show that Ii-Key hybrids can replace one dose of DNA vaccine. Also described in the Exemplification, subsection “Priming Ii-Key peptides augments T cell response in rHA,” mice primed with Ii-Key peptides and boosted with rHA exhibited a significantly stronger response than mice given one or two doses of rHA (FIG. 5B). Indeed priming with one hybrid (Ii-Key 160) induced an IFN-γ response 20 times greater than that of controls. The results show that Ii-Key hybrids can also replace one dose of protein vaccine.

It will be recognized by one skilled in the art that either the Ii-key hybrid construct or the vaccine composition may be administered in the form of a nucleic acid construct encoding an amino-acid-based vaccine or Ii-key construct. A DNA vaccine may be codon optimized to match the codon preferences of the subject. The literature is rich in the description of constructs and methods for the administration of DNA constructs for the purpose of stimulating an immune response with the encoded product. Many such constructs are virus-based, although mechanical methods of introduction (e.g., gene gun technology) can be employed.

H5N1 is a subtype of Influenza A virus. The H5 in H5N1 stands for the fifth of several known types of HA, an antigenic glycoprotein found on the surface of influenza viruses. HA binds the virus to the cell that is being infected. Its name is derived from its ability to cause erythrocytes to clump together. The N1 stands for the first of several known types of neuraminidase, an antigenic glycosylated enzyme also found on the surface of influenza viruses. Hemaglutinin and neuraminidase are the most medically relevant targets for antiviral drugs and antibodies and are thus used as the basis for naming different subtypes of influenza A. The present invention includes methods of enhancing an influenza A vaccine response by priming a subject with Ii-Key hybrid peptides comprised of the LRMK residues and an epitope contained within the hemagglutinin or neuraminidase proteins, followed by boosting with the influenza A vaccine.

Results detailed in the Exemplification section show that mice that were first primed with one of three H5N1 hemagglutinin class II Ii-Key peptides before being boosted with HA DNA vaccine exhibited a greater T cell response than mice that were primed and boosted with HA DNA. The three hybrid peptides tested were peptides 551, 160, and 239. Priming with any one of these hybrid peptides enhances the potency of subsequent HA DNA vaccines. Similarly IFN-γ responses and T cell responses were stronger in mice that were primed with peptides 551, 160, 239, or a combination of the three before receiving a boost of rHA peptide vaccine. As is detailed in the Exemplification, priming mice with Ii-Key 160 and boosting them with rHA induces IFN-γ responses to Ii-Key 160 that are greater than 20 fold higher than in mice receiving two doses of rHA. Priming with any one or a combination of these hybrids enhances the potency of subsequent rHA vaccines. Therefore the present invention includes methods for increasing the potency of an H5N1 influenza DNA or peptide vaccine wherein the MHC class II epitope used in the Ii-Key hybrid comprises HA 551, HA 160, or HA 239. (See Table 1 for sequence.) The data show that one dose of DNA or rHA vaccine can be replaced by Ii-Key peptides.

As discussed in the Background section, pathogens such as H5N1 afflict multiple species of animals. The term ‘mammal’ as used herein is meant to encompass the human species as well as all other mammalian species. The compounds and methods of this invention may be applied in the treatment of diseases and conditions occurring in subjects of all mammalian and bird (fowl and non-fowl) species. The term ‘subject’ as used herein refers to one of any mammalian or bird species, including the human species. The diseases and conditions occurring in humans, and mentioned herein by way of example, shall include comparable diseases or conditions occurring in another species, whether caused by the same organism or pathogenic process, or by a related organism or pathogenic process, or by an unknown or other known organism and/or pathogenic process.

In another embodiment, the present invention pertains to methods of increasing the potency of an HIV DNA vaccine. Results of the HIV DNA Vaccine Experiment described in the Exemplification section show that boosting with Ii-Key hybrids comprising HIV epitopes increases the T cell response of a subsequently administered HIV DNA vaccine. The study shows that mice first primed with a hybrid construct, comprised of Ii-Key's LRMK residues and a Gag protein epitope of the HIV pathogen, before receiving a pcDNA/SynGag booster exhibited a T cell response twice that of mice that were primed and boosted with the DNA vaccine. The present invention includes methods of enhancing the potency of an HIV DNA vaccine wherein the MHC Class II epitope used in the priming step is a Gag epitope. More specifically, the invention includes methods of enhancing the potency of an HIV DNA vaccine wherein the MHC Class II epitope used in the priming step is Gag298.

The present invention further comprises methods of enhancing an HIV DNA vaccine by priming a subject with a hybrid construct of Ii-Key's LRMK residues and the Gag298 epitope, followed by boosting with the DNA vaccine, and subsequently restimulating the subject with an Ii-Key hybrid wherein the MHC Class II epitope comprises the residues of Gag198 or Gag298. (See Table 1 for sequence). Results of the HIV experiment detailed in the Exemplification section show that mice primed with the Ii-Key/Gag298 hybrid, boosted with pcDNA/SynGag, exhibited T cell activity twice as strong as mice which were given two or three doses of DNA.

The present invention also provides a composition for use in priming a DNA or peptide vaccine directed toward a pathogen of interest. The composition comprises an Ii-Key hybrid construct in a pharmaceutically acceptable carrier wherein the construct is comprised of the LRMK residues of Ii-Key protein and a hemagglutinin MHC class II epitope encoded by the H5N1 strain of Influenza A. More specifically the present invention includes a composition wherein the hybrid construct includes the epitope HA 551, HA 160, or HA 239. The results of the Influenza DNA and rHA Vaccine Experiments in the Exemplification section show that mice primed with Ii-Key hybrids composed of epitopes encoded by H5N1 (specifically HA 551, HA 160, or HA 239) exhibit greater CD4 T cell activity than mice primed with HA DNA vaccine.

In another embodiment, the present invention provides a composition comprising an Ii-Key hybrid construct in a pharmaceutically acceptable carrier wherein the construct is comprised of the LRMK residues of Ii-Key protein and a Gag MHC class II epitope encoded by HIV. More specifically the present invention includes a composition wherein the hybrid construct includes the epitopes Gag198 or Gag298. The results of the HIV experiment in the Exemplification section show that mice primed with Ii-Key hybrids composed of epitopes encoded by HIV (specifically Gag298) exhibit greater CD4 T cell activity than mice primed with Gag DNA vaccine. Furthermore, mice primed with an Ii-Key/Gag298 hybrid, boosted with Gag DNA, and then restimulated with an Ii-Key/Gag198 hybrid exhibit a T cell response equivalent to mice receiving three doses of DNA. The present invention includes methods wherein Ii-Key hybrid peptides may also be used to restimulate an immune response.

One skilled in the art, using routine experimental methods, could substitute various natural or non-natural amino acids at respective residue positions in the hybrid peptide. Some examples of molecules which may be substituted are peptidomimetic structures, D-isomer amino acids, N-methyl amino acids, L-isomer amino acids, modified L-isomer amino acids, and cyclized derivatives.

The data show that Ii-Key hybrids can replace at least one dose of DNA; thereby increasing the availability of potential FDA approved DNA vaccines for pathogens such as H5NI influenza. More importantly, stronger T cell responses may contribute to more rapid, greater, and longer-lasting protection.

### Exemplification

Ii-Key technology can augment the immunogenicity of DNA vaccines, including influenza H5N1 and HIV vaccines. Two present DNA vaccine studies have demonstrated that priming the immune system with an Ii-Key hybrid peptide, followed by boosting with a DNA vaccine can augment the overall T cell response in the vaccinated organism. Recombinant Peptide Vaccine Experiments presently described show that priming the immune system with an Ii-Key hybrid peptide can also augment the potency of a peptide vaccine. Using such a heterologous prime/boost method for vaccination should provide long-term protection from pathogenic infection.

### Ii-Key Peptide/DNA Immunization

Mice used in all vaccination experiments were 6-8 week old females of the inbred BALB/c (H-2^{d}) strain (Charles River Laboratories, Wilmington, Mass.) housed under Specific Pathogen Free (SPF) conditions at the university of Massachusetts Medical Center animal facility. All procedures were carried out in accordance with IACUC protocols. Dosing of vaccines and immunization time is defined in each figure legend. In general, mice were sacrificed ˜2 weeks after the last immunization. Their spleens were harvested aseptically and placed into sterile culture medium until homogenization. Serum samples were collected via the retro-orbital plexus and frozen at −20° C. until analysis.

### ELISPOT

IFN-γ and IL-4 ELISPOT cytokine kits (BD Biosciences, San Diego, Calif.) were used to assess immunological responses following immunization with DNA or protein. ELISPOT plates were coated with 5 μg/ml anti-IFN-γ or anti-IL-4 antibody diluted in sterile PBS (100 μl/well) and incubated overnight at 4° C. Plates were washed 1× with 200 μl/well blocking buffer (RPMI 1640/10% FBS/1% penicillin-streptomycin-L-glutamine), followed by addition of blocking buffer (200 μl/well) and incubation at room temperature for 2 hours.

Bulk splenocyte preparations were prepared by homogenization of spleens in 2% complete culture media (RPMI 1640/2% FBS/1% penicillin-streptomycin-L-glutamine), using Dounce homogenizers (Wheaton Science Products, Millville, N.J.), followed by filtration through a 70 μm cell strainer (BD Biosciences, Franklin Lakes, N.J.). To prevent cell clumping, splenocytes were briefly treated with Dnase I (EMD Biosciences, San Diego, Calif.), centrifuged, and RBC's lysed by the addition of Red Cell Lysis Buffer (Sigma, St. Louis, Mo.). 2% complete media was added to inhibit further lysis, followed by centrifugation. The resulting cell pellets were resuspended in 2% complete media at a concentration of 5×10⁶/ml (influenza experiments) or 1×10⁷/ml (HIV experiment).

Following blocking, plates were washed 1× with 2% complete media and decanted. Peptides were resuspended in 2% complete media and added at a concentration of 5 μg/well (100 μl/well). Bulk splenocytes were plated at a concentration of 1×10⁶ cells/well (HIV experiment) or 5×10⁵ cells/well (influenza experiment) in triplicate at 100 μl/well in 2% complete media. Negative controls were composed of media only and unstimulated splenocytes, while phytohemagglutinin/ionomycin-stimulated cells served as a positive control. ELISPOT plates were incubated for 72 hr. at 37° C. with 5% CO₂.

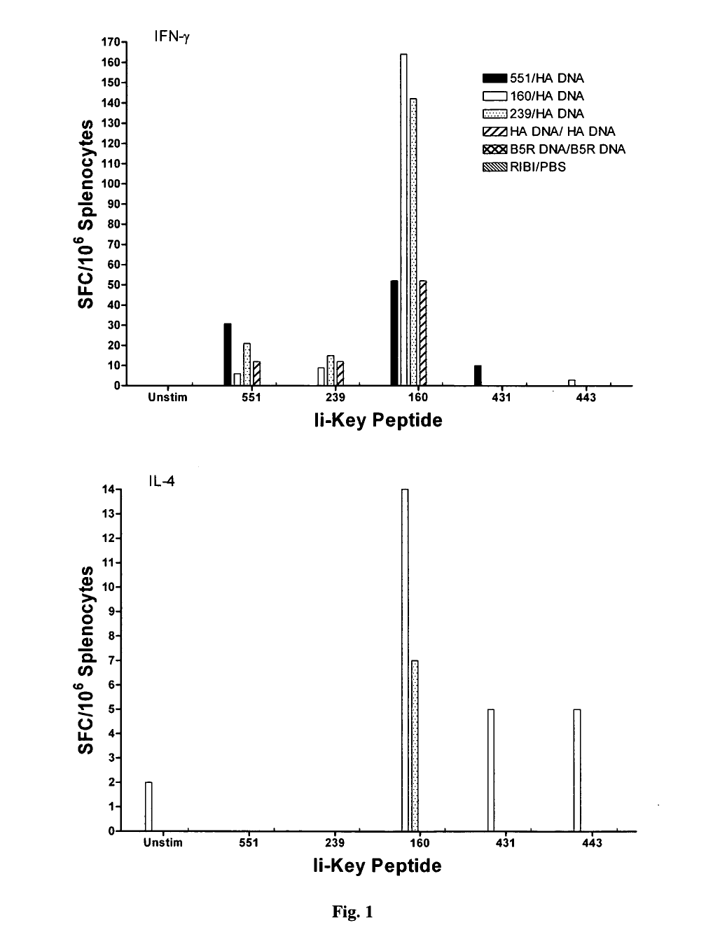

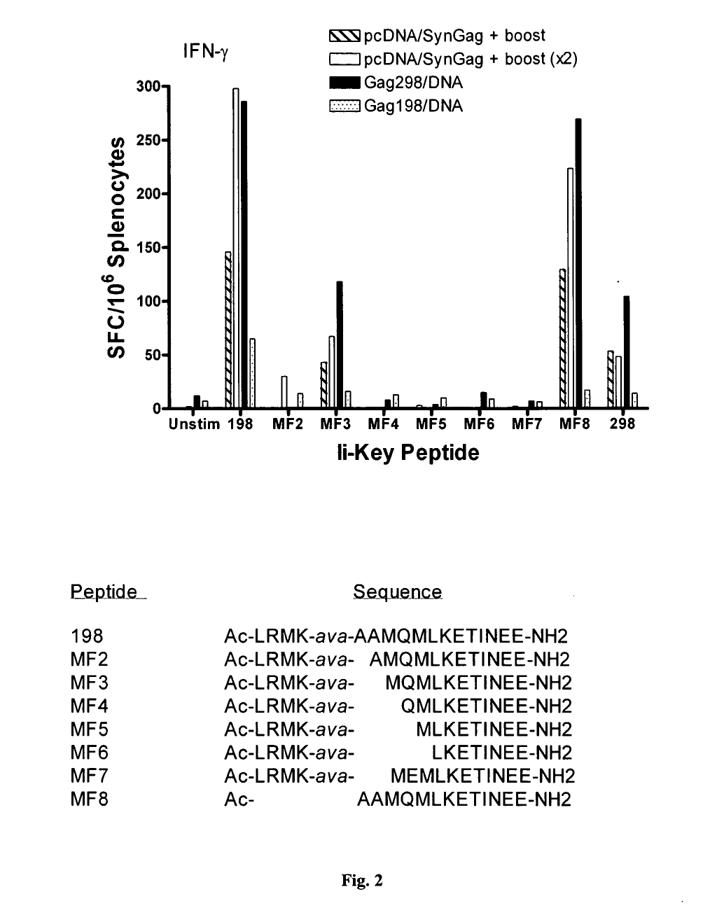

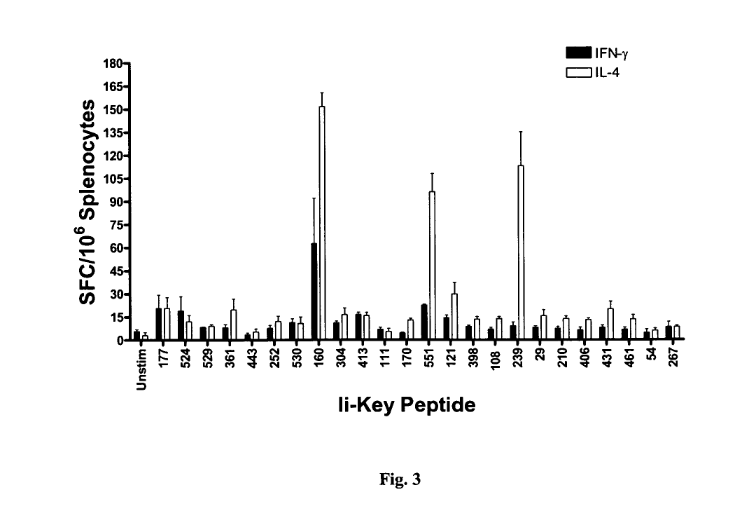

Plates were washed 2× with PBS/0.5% Tween (PBST), followed by the addition of biotinylated anti-mouse IFN-γ or anti-mouse IL-4 antibody (2 μg/ml) diluted in PBS/10% FBS. After a 2 hour incubation, plates were washed 3× with PBST, followed by the addition of streptavidin/HRP (1:100) diluted in PBS/10% FBS. After incubating one hour at room temperature, plates were washed 3× with PBST, followed by two washes with PBS. Assay development was carried out by the addition of AEC substrate (BD Biosciences, San Diego, Calif.) until sufficient spot formation occurred, followed by rinsing with ddH₂O. ELISPOT plates were sent to CTL Analyzers (Cleveland, Ohio) for scanning, followed by data analysis using Immunospot software (CTL Analyzers, Cleveland, Ohio). Data represents the geometric mean (FIGS. 1-2) or the mean±Standard Error of the Mean (SEM) (FIGS. 3-7) of each Ii-Key peptide tested in triplicate.

CD4+ depletion of bulk splenocyte preparations was performed by negative selection using MACS® cell separation columns according to the manufacturer's directions (Miltenyi Biotec, Auburn, Calif.). Depleted cell populations were plated and restimulated identically as described above.

### Influenza DNA Vaccine Experiment

At week 0, mice (5/group) were primed with one of three H5N1 hemagglutinin (HA) class II Ii-Key peptides, designated 551, 160, or 239; followed by boosting with HA DNA vaccine at week 2 (FIG. 1). Control mice were primed and boosted with either an irrelevant DNA vaccine (B5R) or RIBI followed by PBS. The mice were sacrificed twelve days later and the splenocytes of each treatment group were pooled. In vitro peptide restimulation assays using the three Ii-Key peptides above were then performed on each pool of splenocytes. The results showed that the overall IFN-γ response, which is a marker for CD4 T cell activity, was most profound when mice were primed with either the 551, 160, or 239 peptide compared to mice that received two DNA vaccine doses. Interleukin (IL)-4 responses, a measure of CD4 T cell activation, in mice primed with peptide 160 and boosted with HA DNA were strongest when splenocytes were restimulated with the 160 hybrid peptide. Mice which received two doses of DNA did not induce detectable IL-4 responses. The data suggest that Ii-Key hybrids can replace one dose of DNA.

### HIV DNA Vaccine Experiment

Gag is a protein involved in the assembly of HIV-1 virus particles. At week 0, mice were primed with either pcDNA/SynGag, HIV Gag298, or Gag198 Ii-Key peptide; followed by boosting with pcDNA/SynGag vaccine on day 9 (FIG. 2). One group of pcDNA/SynGag primed and boosted mice was boosted a second time with pcDNA/SynGag on day 16. Mice were sacrificed eleven days post-boost and the splenocytes of each treatment group were pooled. In vitro peptide restimulation assays using Ii-Key peptides were performed on each pool of splenocytes. IFN-γ responses demonstrate that T cell responses can be further improved by priming with Ii-Key peptides. Although there appears to be no benefit of priming with Gag198, relative to DNA immunized mice, there was an unexpected finding that mice primed with Gag298 and restimulated in vitro with Gag198 yielded T cell responses that were as strong as mice receiving a total of three doses of DNA. This suggests that Gag298 is a very promiscuous class II epitope and/or that Gag298 has immunological “bystander” activity. Mice primed with Gag298 and boosted with DNA vaccine, followed by restimulation in culture with Gag298, revealed IFN-γ responses that were approximately two-fold stronger than either of the DNA vaccine regimens (one boost or two boosts). IFN-γ response in HIV infected individuals is only one of several parameters used to monitor disease progression and has been shown to be important in early stages of infection. In addition, the importance of CD4+ T cells in controlling viral replication cannot be overstated and as such, using Ii-Key hybrids in conjunction with an HIV DNA vaccine should improve the frequency, duration, and strength of T cell immunity.

### Recombinant Peptide Vaccine Experiments

### Ii-Key Peptide/rHA Immunization

Mice (4/group) were individually immunized with 100 μg Ii-Key peptides or 33 μg/peptide (in combination), followed by boosting with 10 μg rHA (Protein Sciences, Meriden, Conn.) at day 21. Both vaccine components were emulsified in RIBI adjuvant (Sigma, St. Louis, Mo.) and injected subcutaneously at the base of the tail, with the exception of one experiment whereby rHA was emulsified in CFA/IFA (FIG. 3). DNA immunized mice received 50 μg pcDNA/HA delivered intramuscularly, followed by boosting at Day 14.

### Peptide Design and Synthesis

The preferred method for identifying H5N1 HA class II epitopes is to obtain Peripheral Blood Mononuclear Cells (PBMC) from individuals previously exposed to the antigen of interest and screen them against predicted or overlapping epitopes. Since PBMC from individuals exposed to the H5N1 virus or to an H5N1 vaccine in development are very difficult to obtain, an alternative strategy to identify potential human recognized MHC class II epitopes was employed.

SYFPEITHI (www.syfpeithi.de) maintains a database of >450 peptide sequences known to bind MHC class I and II molecules and provides algorithms useful for predicting epitopes. The SYFPEITHI algorithm was used to maximize the likelihood of identifying promiscuous HA epitopes from the H5N1 A/Duck/Anyang/AVL-1/2001 amino acid sequence (GenBank; accession #AF468837). Epitopes were predicted for HLA-DRB 1 alleles (DRB 1*0101, DRB I*0301, DRB 1*0401, DRB I*0701, DRB I*1101, and DRB I*1501). The 40 top-scoring predicted epitopes were ranked on a cumulative basis according to the score reported from the SYFPEITHI program for the alleles indicated. Applying additional criteria and constraints to the top 40 scoring peptides resulted in a smaller panel of 24 class II epitopes to test. The selection criteria have been previously described in greater detail (http://www.pharmadd.com/archives/May%2016%20%202006/BN%20Tech%20Brief.asp).

Peptides were synthesized (NeoMPS, San Diego, Calif.) and modified to incorporate the Ii-Key motif (LRMK), which was covalently Iinked to the N-terminus of each epitope via a Iinker sequence (5-aminopentannoic acid, ava). Previous experiments have demonstrated that one ava Iinker is optimal for T cell induction (Kallinteris, N. L. et al., Frontiers in Bioscience: 46 (2006)). Peptides were dissolved in 20% DMSO and frozen at −80C until use.

Prior studies using Ii-Key hybrids of a promiscuous HER-2/neu epitope, which induces robust antigenic-specific CD4+ stimulation in humans, also demonstrated strong CD4+ activation in BALB/c mice (unpublished observations), indicating that this animal model might also serve to characterize Ii-Key hybrids of promiscuous H5NI HA epitopes. Of 24 HA epitopes predicted on a cumulative basis to interact with six of the most common human alleles, three were found which were recognized by CD4+ cells from immunized BALB/c mice.

### ELISA Analyses

Antibody responses were measured by indirect ELISA assay. Maxisorp plates (Nunc, Rochester, N.Y.) were coated with either rHA purified protein (Protein Sciences, Meriden, Conn.) or BSA at 1 μg/ml in coating buffer. Plates were incubated overnight at 4° C., then washed 3× with PBST using a Wellwash 4 Mk2 plate washer (Thermo Labsystems, Waltham, Mass.), followed by blocking (PBS, 3% BSA, 0.01% thimerosal) for 1 hour at room temperature. Sera samples (six Log₂ dilutions) were diluted in diluent (PBS, 0.1% Tween 20, 0.1% BSA, 0.01% thimerosal), plated for 2 hours at room temperature, and washed 3×. Goat anti-mouse total IgG (Vector Labs, Burlingame, Calif.) was diluted 1:8,000 or biotinylated rat anti-mouse IgGI or IgG2a at 2 μg/ml (BD Biosciences, San Jose, Calif.) and plated for 1 hour at room temperature, followed by washing and the addition of streptavidin/HRP, diluted 1:2,000 (Vector Labs, Burlingame, Calif.). After incubating 30 minutes, plates were washed 4×, followed by the addition of TMB substrate solution (BD Biosciences, San Jose, Calif.). Once sufficient color development occurred, 2N H₂SO₄ was added to stop the reaction. Signal intensity was measured using an LD400 ELISA plate reader (Beckman Coulter, Fullerton, Calif.). End point titrations were determined by using non-linear regression analysis (Graphpad Prism, San Diego, Calif.) in which the OD₄₅₀ nm≧2× over that of naive animals' sera at a dilution yielding an OD₄₅₀ nm of ≧0.2.

### Screening Human Predicted MHC Class II Epitopes with rHA Immunized Mice

Immunizing with rHA permits the natural processing and presentation of class II epitopes. APC/MHC Class II presentation results in the induction of T cells that may recognize some of the 24 candidate peptides identified by the SYFPEITHI epitope predicting program. In order to screen human predicted MHC class II epitopes for recognition by the BALB/c H-2^{d} allele, BALB/c H-2^{d} mice were first immunized with 10 μg of rHA emulsified in CFA, and then boosted two weeks later with 10 μg of rHA emulsified in IFA. Rather than testing each peptide individually for immunogenicity or in the context of rHA, it was more practical to immunize mice first with rHA, restimulate bulk splenocytes with each of the 24 peptides and determine which peptides were recognized. Restimulating with Ii-Key peptides 160, 551, and 239 yielded the strongest IFN-γ and IL-4 responses, with lower, but detectable responses observed towards other peptides in the panel (FIG. 3). Since all the peptides were predicted to bind several human Diversity Region (DR) alleles, it was not surprising that only 3 out of 24 were recognized by BALB/c mouse splenocytes after immunization with rHA in the presence of a strong parenteral adjuvant. Low affinity of some of the peptides for the class II groove has been suggested to induce suboptimal T cell activation (Gregers, T. F. et al., Int Immunol 15:1291 (2003)) and may have caused the decreased frequency of recognition.

### Screening Human Predicted MHC Class II Epitopes with DNA Immunized Mice

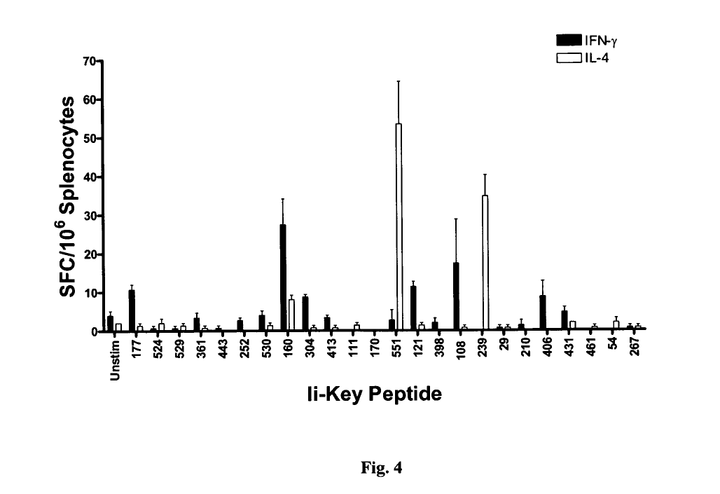

To further substantiate the immunogenic activity of Ii-Key /epitope hybrids 160, 551, and 239, T cell responses were measured in BALB/c mice immunized with pcDNA/HA. This plasmid vaccine expresses full length HA (sequence derived from A/Duck/Anyang/AVL-1/2001) and would allow for further characterization of epitopes recognized in mice. Mice were immunized intramuscularly at day 0 and day 14 with 50 μg DNA, followed by in vitro peptide restimulation. Ii-Key peptides 160, 551 and 239 again were most frequently recognized and induced the strongest responses, although moderately skewed towards an IL-4 response (FIG. 4). Since the same peptides were strongly recognized in both the DNA and rHA and immunized mice (FIG. 3), it was determined that peptides 160, 551 and 239 would likely be the most immunogenic for performing subsequent prime/boost studies with rHA.

### Immunization with Ii-Key Peptides Induces IFN-γ Responses

Because CFA emulsification is expensive and requires the use of more rHA, the RIBI adjuvant system was used subsequently. RIBI requires much less rHA during vaccine preparation and is less toxic than CFA. To confirm the immunogenicity of the identified epitopes, BALB/c mice were immunized subcutaneously at day 0 with 100 μg of Ii-Key peptides 160, 551 or 239 individually emulsified in RIBI, and boosted at day 14. Mice immunized with two doses of either Ii-Key peptides 551 or 160, followed by restimulation with the same peptides induced moderate IFN-γ responses, with the response to 160 being most prominent (FIG. 5A). Immunization with Ii-Key peptide 239 induced only weak but detectable levels of IFN-γ during restimulation with the 239 peptide. However, there appeared to be cross-recognition to the Ii-Key 160 peptide with the T cells of Ii-Key 239 immunized animals. Mice in this group exhibited a modest response when restimulated with Ii-Key 160 peptide, possibly from a broadened T cell subset cross reacting with the Ii-Key 160 peptide. There were no detectable IL-4 responses from mice immunized with the RIBI/RIBI regimen. Studies in which mice were only immunized once, at day 0 with boost, were also carried out. One dose of Ii-Key 160 peptide resulted in decreased levels of IFN-γ relative to two doses of Ii-Key 160. One dose of either 551 or 239 resulted in undetectable levels of IFN-γ (data not shown). The results indicate that at least Ii-Key 160 is immunogenic after only one immunization.

### Priming with Ii-Key Peptides Augments the T Cell Response to rHA

Clinical studies have shown that the same rHA used in these studies was poorly immunogenic even in individuals receiving two 90 μg doses of rHA (Treanor, J. J., et al., Vaccine 19:1732 (2001)). Investigations were carried out to measure whether priming with class II epitopes enhances T cell immunogenicity towards rHA. In the first study, at Day 0, mice were primed with 100 μg of Ii-Key peptide 239, 551 or 160 individually or 33 μg of each peptide in combination, followed by boosting at Day 21 with 10 μg of rHA emulsified in RIBI adjuvant. Control mice were primed with RIBI adjuvant, followed by rHA. In vitro restimulation was performed two weeks post-boost with either the Ii-Key peptides used in the immunization or the Antigenic Epitope Alone (AEO, class II epitope without Ii-Key moiety). Hybrid and AEO peptides induced strong IFN-γ responses following a 48 hr restimulation (FIG. 3B). In general, all groups primed with Ii-Key peptides induced T cell responses to at least the Ii-Key 160 peptide. They also induced responses to 160AEO, showing that the response was primarily directed against the 160 epitope and not the attached Ii-Key region or a potential junctional epitope. Relative to mice immunized with the RIBI/rHA and rHA/rHA regimens, the strongest and most significant responses were observed in animals primed with Ii-Key 160 peptide and boosted with rHA (160/rHA, P<0.001), followed by peptides used in combination (239,551,160/rHA, P<0.001) and then 239/rHA (P<0.05) and 551/rHA (P>0.05). Again, mice primed with Ii-Key 239 and restimulated with Ii-Key 160 appeared to have T cells which cross-reacted with the 160 epitope as was previously observed, although no sequence homology exists between the two epitopes (FIG. 3A.). The significant induction of IFN-γ relative to the RIBI/rHA regimen, confirmed the ability of Ii-Key peptides, namely 160 and those used in combination, to prime the T cell response. No detectable levels of IL-4 were measured in any group, suggesting there was suboptimal priming of Th2 cells or possibly a lack of detectable IL-4 following a 48 hr peptide restimulation. The decreased response in mice primed with all three peptides in combination is likely due to those mice receiving only 33 μg/peptide, as opposed to 100 μg/peptide for all other experimental groups. Responses to in vitro restimulation with either 239 or 551 induced only weak or non-detectable responses regardless of the heterologous prime/boost regimen.

The aim of a second study was to compare the T cell response of mice receiving an Ii-Key peptide/rHA vaccination regimen to those receiving two doses of rHA (rHA/rHA). Mice immunized with two 10 μg doses of rHA emulsified in RIBI adjuvant yielded undetectable to low IFN-γ responses to all peptides tested under in vitro restimulation conditions. The results were the same in mice that received one (RIBI/rHA) or two doses (rHA/rHA) of rHA. This is in contrast to the much stronger responses observed when mice were immunized with rHA emulsified in CFA/IFA (FIG. 1.), suggesting that the less potent RIBI adjuvant may have induced suboptimal T cell responses. Mice primed with Ii-Key 160, boosted with rHA, and restimulated with Ii-Key 160 exhibited IFN-γ responses >20 fold higher than mice receiving two doses of rHA.

To determine whether the rHA boost was important and to rule out the possibility that the phenomenal response induced by the 160/rHA regimen was due to the Ii-Key 160 priming step alone, studies were performed in which mice were primed with the Ii-Key 160 peptide without an rHA boost. Reduced, but detectable, IFN-γ responses (data not shown) resulted. This suggests that in the context of a heterologous immunization, the rHA booster immunization does contribute towards the overall T cell response, but that the rHA immunization alone results in poor T cell immunogenicity. It is possible that the higher molar amounts of epitope resulting from priming with 100 μg of Ii-Key 160 provides sufficient MHC class II loading to initiate an antigen-specific T cell response which can be augmented by boosting with rHA, and that rHA alone provides far fewer MHC class II/peptide complexes resulting in less efficient priming. It is also plausible that the 160 epitope generated by natural processing and presentation is not completely identical in sequence to the algorithm-predicted 160 Ii-Key peptide, These potential differences may have an effect on both the T cell affinity for the epitope and subsequent induction of T cell responses. However, experiments have shown that bulk splenocytes from mice immunized with the 160/rHA regimen and restimulated with rHA had a higher frequency of IFN-γ secreting T helper cells compared to mice that received two doses of rHA, confirming the ability of the Ii-Key 160 peptide to induce responses that recognize naturally processed and presented 160 from native protein (data not shown). These data support the concept that one dose of rHA can be replaced (in a two-dose regimen) by priming with Ii-Key/epitope hybrid peptide vaccines and that limited supplies of rHA or other vaccines could thus be increased during a pandemic.

Highly conserved MHC class II H5N1 HA epitopes that are predicted to bind multiple HLA alleles with high affinity have been modified to include Ii-Key. This modification permits extracellular loading and charging of MHC class II molecules, followed by direct stimulation of CD4+ T cells. As a “stand alone” vaccine, Ii-Key peptides can be used to prime naive T cells, which following infection may provide sufficient preexisting immunity to alter the course and severity of viral infection. Subsequent experiments have demonstrated that immunization of mice with Ii-Key/H5 hybrids alone lead to strong T cell responses. The induction of the greatest levels of protective immunity will most likely result from the utilization of Ii-Key peptides with other subunit or inactivated viral vaccines already in development. Priming with Ii-Key hybrids provides a strong T cell response that is not only highly specific, but also provides broad T cell help by way of cytokine induction and the potential for inducing heterosubtypic immunity against other H5 variants. Thus, it is expected that the CD4+ T cells stimulated by Ii-Key hybrids will provide some level of immunity against emerging H5N1 strains that would otherwise not be present. Finally, the use of peptide vaccines has several advantages over traditional vaccines including a relatively long shelf life, ease of manufacturing and a good safety profile in clinical trials.

### IFN-γ Responses are Dependent on CD4+T-Helper Cells

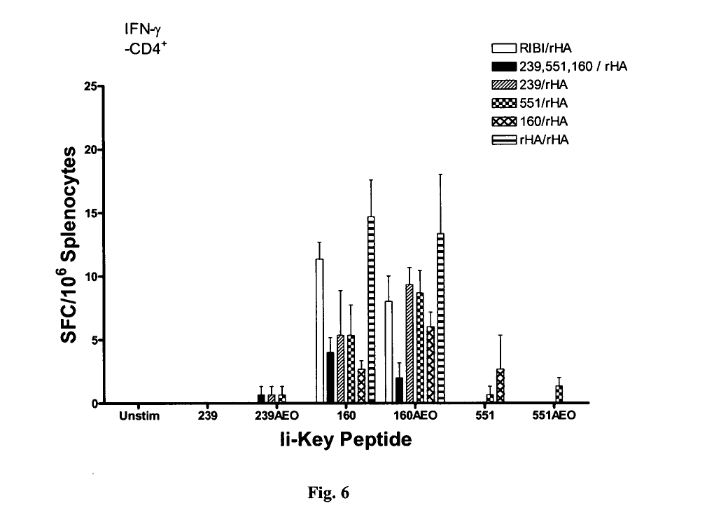

It is believed that Ii-Key peptides charge MHC class II molecules at the surface of APC's and activate naive T-helper cells. The cytokine IFN-γ can be secreted from various cell types, including Th1, NK and CD8+ cells. Experiments were performed to verify the source of IFN-γ in the in vitro restimulation assays described above. It was expected that most of the IFN-γ secretion was derived from T-helper cells. Therefore, splenocytes were depleted of CD4+ T cells, followed by restimulation with each peptide. After a 48 hr restimulation, levels of IFN-γ in all groups were greatly reduced in the absence of CD4+ T cells (FIG. 6), confirming that the primary source of this cytokine was T cell derived (Th1), although the possibility of low-level NK or CD8+ T cell activity cannot be ruled out.

### Hemagglutination Inhibition (HI) Determination

HI analysis, using the VNH5N1-PR8/CDC-rg virus, was performed by Southern Research Institute (Birmingham, Alab.). Diluted serum samples were treated with RDE (Seiken Denka, Japan) overnight at 37° C., followed by inactivation at 56° C. for 30 minutes. Sera samples were then serially diluted, followed by the addition of the VNH5N1PR8/CDC-rg virus (4 HAU/well). After a 30 minute incubation at room temperature, 1% horse red blood cells (RBC) were added, followed by an additional 1 hour incubation. HI titers were determined by visual analysis and reported as the reciprocal dilution which contained pelleted RBC's.

### Heterologous Immunization with Ii-Key Peptides can Augment the Humoral Response and Induce HI Activity

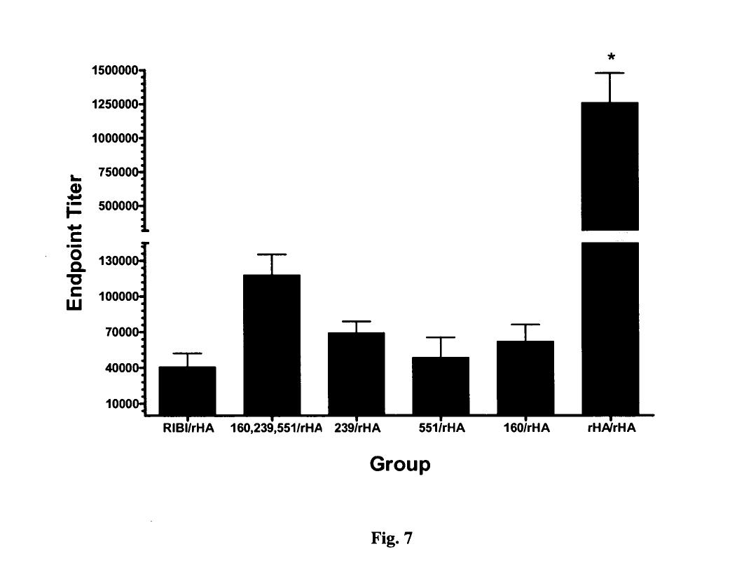

The presence of high-titered viral specific antibodies capable of neutralizing virus plays a crucial role in individuals that have survived H5N1 infection (Katz, J. M. et al., J Infect Dis 180:1763 (1999)) and other influenza A viral infections (Mozdzanowska, K. et al., J Virol 79:5943 (2005)). Studies have shown that priming with highly conserved T-helper epitopes prior to recombinant protein immunization can enhance the antibody response to protein vaccines (Hosmalin, A., P. et al., J Immunol 146:1667 (1991); Vaslin, B. et al., AIDS Res Hum Retroviruses 10:1241 (1994)). Studies related to the present invention show that priming with Ii-Key MHC Class II peptides potentiates the B cell response and induces enhanced antibody responses via T cell help. Mice were primed separately with either Ii-Key peptide 239, 551, or 160, or RIBI (control), followed by an rHA booster. The Ii-Key hybrid/rHA regimens resulted in a modest, but non-significant increase in total IgG against rHA (titers 5×10⁴) relative to control mice (FIG. 7). Mice were also immunized at day 0 with a combined peptide emulsification (33 μg/peptide) and boosted at day 21 with rHA to determine if there was a synergistic effect of combining the three peptides. Interestingly, there was an increase in overall IgG titer relative to priming with each Ii-Key peptide individually, suggesting a possible synergistic effect of combining the three peptides. Mice receiving two doses of adjuvanted rHA yielded very strong IgG responses, consistent with other similar studies (Hoelscher, M. A. et al., Lancet I))OI:10.1016/S0140-6736(06)):68076 (2006)). Giving large doses of rHA may lead to the recruitment of only low affinity B cells which recognize surface exposed B cell epitopes of the HA protein. By priming with peptides, more potent T cell clones may be induced, which in turn stimulate B cells with surface immunoglobulin recognizing lower concentrations of rHA, potentially leading to antibodies of higher affinity.

To determine whether priming with Ii-Key peptides would skew the antibody response towards a Th1 (IgG2a) or Th2 (IgGI) type response, sera samples were tested for the presence of anti-HA IgGI and IgG2a antibody. Because of the results in the ELISPOT analysis described above, in which only IFN-γ responses were detected in RIBI-adjuvanted experiments, higher titers of IgG2a were expected, indicative of a Th1 polarized response. Contrary to expectations, most samples elicited a Th2-like response, yielding higher IgGI anti-HA titers (Table 3). Interestingly, mice primed with either the 551 or 160 peptide appeared to have a suppressed IgG2a response, relative to RIBI/rHA. However mice primed with 239/rHA had greater IgG2a titers, implying that 239 may have some immunomodulatory activity at the B cell level even though 239 was not observed to induce strong T cell-derived IFN-γ responses following 239/rHA immunization. Combining all three peptides as a priming strategy induced similar levels of both isotypes and in general induced the highest of the peptide-primed titers, which was consistent with the total IgG end-point titers. The fact that the serum isotype responses did not correlate with the T cell responses may be influenced by the use of RIBI adjuvant. Other studies have shown increased levels of IgG1 in the presence of RIBI (Fogg, C. et al., Virol 78:10230 (2004)) and this may in part explain the disparity observed here.

To examine more fully the functionality of the antibody response, sera samples were tested for HI activity (performed at SRI). Samples were treated with antiserum with receptor destroying enzyme and then tested for the ability to prevent the agglutination of horse red blood cells after incubation with inactivated VNH5NI-PR8/CDC-rg surrogate virus. This virus is antigenically identical to the Vietnam strain (A/VN/1201/03), but is non-pathogenic. It was expected that the group having the highest IgG end-point titer would also have the strongest HI titer, consistent with the observations made in the total IgG analysis. Mice primed with a combined Ii-Key peptide regimen induced HI titers that were more than 6-fold higher [320 geometric mean] than the control group, which yielded marginal HI activity [50] (Table 2). Priming with Ii-Key 239, 551, or 160 individually induced titers of 202, 101, and 202 respectively. Although mice receiving two doses of rHA yielded the highest HI titer [2032], it was interesting to note that relative to the RIBI/rHA group, one rHA dose induced much lower titers [50], suggesting that one rHA dose is suboptimal for B cell induction. Although HI titers of >40 in humans are thought to be protective based on serological surveys performed on H5N1 infected and uninfected persons (Katz, J. M. et al., J Infect Dis 180:1763 (1999)), such a titer does not guarantee protection, as people with higher titers can have symptomatic infection (Poland, G. A., N Engl J Med 354:141 (2006)).

To circumvent the inadequate immunogenicity and supply issues surrounding current vaccines, we have demonstrated that as a priming strategy, the utilization of modified MHC class II epitope vaccines can increase the T and B cell responses to rHA and possibly to other conventional or alternative vaccines in development. Such an approach to vaccination may reduce the strain on the manufacture of traditional vaccines (e.g. egg-based); improve their effectiveness and increase the number of available vaccine doses. The present studies demonstrate that by priming with MHC class II epitopes, T cell responses are markedly improved over one or two doses of rHA. ELISPOT analysis revealed a predominance of Th1-derived IFN-γ, with no detectable levels of IL-4, suggesting a Th1 polarized response. IFN-γ secretion from CD8+ cells has been shown to protect against lung tissue damage (Wiley, J. A. et al., Am J Pathol 158:119 (2001)), while Th2 induction exacerbated tissue destruction. In addition, studies have demonstrated that Th2 cells do not protect mice against lethal challenge (Graham, M. B. et al., J Exp Med 180:1273 (1994)). Therefore, Ii-Key peptides, in conjunction with rHA, which induce a Th I-polarized response, may be advantageous for the treatment of infection while minimizing lung tissue damage.

Conversely, antibody responses from these studies did not entirely correlate with T cell responses. Mice receiving 160/rHA and 551/rHA vaccine regimens induced higher levels of IgGI, indicative of a Th2 response, even though there was an absence of detectable IL-4 during ELISPOT analysis, while remaining groups in the study resulted in more evenly distributed IgGI/IgG2a titers. Although two doses of rHA yielded much higher IgG end-point and HI titers, one dose yielded considerably lower overall serum titers, suggesting that induction of high-titered antibody responses requires high doses of antigen. Priming with a combination of all three Ii-Key peptides augmented the B cell activity towards rHA, relative to one rHA dose. The results demonstrate that one rHA dose can be replaced by Ii-Key hybrid peptides, thereby increasing vaccine supply. This finding also suggests that the combination of T-helper epitopes induces additional CD4+ activity that non-specifically provides help to B cells for the production of antibodies as has been suggested by others (Horvath, A. et al., Immunol Lett 60:127 (1998); Ninomiya, A. et al., Vaccine 20:3123 (2002)).

### Statistics

Statistical analysis was performed where appropriate with GraphPad Prism, v. 4.03 (GraphPad, San Diego, Calif.). Data was analyzed using ANOVA analysis and Newman-Keuls multiple comparison test. Differences between groups were considered significant if P<0.05.

Studies presented here demonstrate the potential utility of Ii-Key/epitope hybrid peptide vaccines as a means to prime the immune system and thereby increase immunological responsiveness to HIV or influenza vaccines and/or to the virus itself.

TABLE I
H5N1 HA and HIV Gag Ii-Key Peptides used for immunization

| Peptide | Linker^{α} | Sequence^{ψ} |
|---|---|---|
| Ii-Key HA239 | LRMK-ava | GQSGRMEFFWTIL |
| Ii-Key HA551 | LRMK-ava | VAGLSLWMCSNGS |
| Ii-Key HA160 | LRMK-ava | SFFRNVIWLIKKN |
| Ii-Key Gag198 | LRMK-ava | AAMQMLKETINEE |
| Ii-Key Gag298 | LRMK-ava | YVDRFYKTLRAEQ |

^{α}Ii-Key (LRMK) is covalently linked to N-terminus of peptide sequence via ava (5-aminopentanoic acid) spacer.
^{ψ}Core sequence denoted in bold.

TABLE 2
Hemagglutination inhibition titers against VNH5N1-PR8/CDC-rg

| | HAI Titer^{α} (replicate #) | | | |
|---|---|---|---|---|
| Vaccine^{ψ} | 1 | 2 | 3 | GMT |
| RIBI/rHA | 80 | 40 | 40 | 50 |
| 239/rHA | 320 | 160 | 160 | 202 |
| 551/rHA | 160 | 80 | 80 | 101 |
| 160/rHA | 160 | 160 | 320 | 202 |
| 239, 551, 160/rHA | 320 | 320 | 320 | 320 |
| rHA/rHA | 2560 | 2560 | 1280 | 2032 |

Pos >2560 (CDC reference H5N1 antisera)
Neg 10 (Human H5N1 negative sera)
^{α}Reciprocal HAI dilution
^{ψ}Mice were immunized subcutaneously at day 0 with 100 μg, 33 μg/peptide or 10 μg rHA emulsified in RIBI adjuvant, followed by boosting at day 21 with 10 μg rHA. Two weeks post-boost, anti-sera was collected and assayed for HI activity against VNH5N1-PR8/CDC-rg (performed at Southern Research Institute).

TABLE 3
Mouse serum anti-HA isotype responses following immunization

| | Isotype End Point Titer^{α} | | |
|---|---|---|---|
| Vaccine^{ψ} | IgG2a | IgG1 | (IgG2a/IgG1) |
| RIBI/rHA | 37,000 ± 9,000 | 41,000 ± 13,000 | 0.90 |
| 239/rHA | 82,000 ± 14,000 | 128,000 ± 51,000 | 0.64 |
| 551/rHA | 8,000 ± 3,000 | 41,000 ± 20,000 | 0.20 |
| 160/rHA | 13,000 ± 4,000 | 75,000 ± 22,000 | 0.17 |
| 239, 551, | 116,000 ± 16,000 | 116,000 ± 14,000 | 1.00 |
| 160/rHA | | | |
| rHA/rHA | 707,000 ± 140,000 | 1,100,000 ± 284,000 | 0.64 |

^{α}Data represents the mean ± SEM
^{ψ}Mice were immunized subcutaneously at day 0 with 100 μg, 33 μg/peptide or 10 μg rHA emulsified in RIBI adjuvant, followed by boosting at day 21 with 10 μg rHA. Two weeks post-boost, anti-sera was collected and assayed for anti-HA IgG2a or IgG1 antibody responses.

What is claimed is:

1) A method for increasing the potency of a vaccine directed toward a pathogen of interest in a subject, the method comprising:
a) providing a vaccine, the vaccine comprising an epitope-containing protein or portion thereof which is encoded by the genome of the pathogen, or a DNA encoding the same;
b) providing an Ii-key hybrid construct comprising:
i) the LRMK residues of Ii-key protein; and
ii) an MHC class II epitope contained within the protein or portion thereof of step a); or DNA encoding the elements of i) and ii).
c) priming the immune system of the subject by administering the Ii-key construct of step b) under conditions appropriate for the stimulation of an immune response in the subject;
d) administering the vaccine of step a) under conditions appropriate to boost the immune response of step c) thereby increasing the potency of the vaccine relative to a non-primed administration.

2) The method of claim 1 wherein the protein of step a) is produced by recombinant DNA technology.

3) The method of claim 1 wherein the vaccine is a DNA vaccine and the codon usage is optimized to match the codon preferences of the subject.

4) The method of claim 1 wherein the pathogen is a virus.

5) The method of claim 1 wherein the pathogen is a bacteria.

6) The method of claim 1 wherein the subject is a mammal.

7) The method of claim 1 wherein the subject is a human.

8) The method of claim 1 wherein the subject is a bird.

9) The method of claim 8 wherein the bird is a fowl.

10) The method of claim 1 wherein the pathogen is Influenza A.

11) The method of claim 10 wherein the protein of step a) is hemagglutinin.

12) The method of claim 10 wherein the protein of step a) is neuraminidase.

13) The method of claim 10 wherein the Influenza A pathogen is strain H5N 1.

14) The method of claim 13 wherein the protein of step a) is hemagglutinin.

15) The method of claim 13 wherein the protein of step a) is neuraminidase.

16) The method of claim 14 wherein the MHC class II epitope comprises the residues GLSLWMCSN.

17) The method of claim 14 wherein the MHC class II epitope comprises the residues FRNVIWLIK.

18) The method of claim 14 wherein the MHC class II epitope comprises the residues SGRMEFFWT.

19) The method of claim 1 wherein the pathogen is HIV.

20) The method of claim 19 wherein the protein of step a) is gag.

21) The method of claim 20 wherein the MHC class II epitope comprises the residues DRFYKTLRA.

22) The method of claim 21 further comprising, between steps c) and d), restimulating the subject with an Ii-key hybrid comprising the LRMK residues of Ii-Key protein and the MHC class II epitope gag198.

23) The method of claim 21 further comprising, between steps c) and d), restimulating the subject with an Ii-key hybrid of step b) wherein the MHC class II epitope comprises the residues DRFYKTLRA.

24) A composition for use in priming a vaccine directed toward a pathogen of interest, the composition comprising an Ii-key hybrid construct in a pharmaceutically acceptable carrier, the Ii-key hybrid construct comprising:
a) the LRMK residues of Ii key protein; and
b) a hemagglutinin MHC class II epitope encoded by the H5N1 strain of Influenza A;
or DNA encoding the elements of a) and b).

25) The composition of claim 24 wherein the MHC class II epitope comprises the residues GLSLWMCSN.

26) The composition of claim 24 wherein the MHC class II epitope comprises the residues FRNVIWLIK.

27) The composition of claim 24 wherein the MHC class II epitope comprises the residues SGRMEFFWT.

28) The composition of claim 24 wherein the Ii-key hybrid construct comprises a DNA encoding the elements of a) and b) and wherein the codon usage is optimized to match the preferences of a subject.

29) A composition for use in priming a DNA vaccine directed toward a pathogen of interest, the composition comprising an Ii-key hybrid construct in a pharmaceutically acceptable carrier, the Ii-key hybrid construct comprising:
a) the LRMK residues of Ii key protein; and
b) a gag MHC class II epitope encoded by HIV;
or DNA encoding the elements of a) and b).

30) The composition of claim 29 wherein the MHC class II epitope comprises the residues DRFYKTLRA.

31) The composition of claim 29 wherein the MHC class II epitope comprises the residues MQMLKETIN.

32) The composition of claim 29 wherein the Ii-key hybrid construct comprises a DNA encoding the elements of a) and b) and wherein the codon usage is optimized to match the preferences of a subject.
